(19) United States
(12) Patent Application Publication
(10) Pub. No.: US 2013/0123917 A1
(43) Pub. Date: May 16, 2013

(54) Breast prosthesis form

(71) Applicant: Christina Marie Riad, McComb, MS (US)

(72) Inventor: Christina Marie Riad, McComb, MS (US)

(21) Appl. No.: 13/373,346

(22) Filed: Nov. 12, 2011

(51) Int. Cl. A61F 2/52

(52) U.S. Cl. CPC A61F 2/52
USPC 623/7

(57) ABSTRACT

A prosthetic breast form and a method of making thereof provides for a flexible resilient stretchable soft pouch adapted to be filled with a desired quantity of granular material to simulate shape and volume of a natural woman's breast. The pouch can be made from a nylon net, spandex, and microfibers, mixed together or with natural fibers. The granular filler can be made from a natural or artificial material. The method provides for the user to fill the pouch with the desired quantity of the filler material suitable for a particular lingerie item or garment.

### BACKGROUND OF THE INVENTION

Breast cancer often results in mastectomy (removal of the breast). One of the options following the procedure is reconstructive surgery; another option is to use breast prosthesis. Even when choosing the reconstructive surgery, patients have to wait more than six months before the breast reconstruction can be made, allowing the tissue sufficient time to heal. In such cases, mastectomy products, such as external breast forms are needed. Prosthesis offers various advantages, such as protection of the patient's chest and surgical scars; prosthesis helps balance the posture and keeps the bra from shifting side to side or riding up. Importantly, prosthesis helps prevent problems with curvature of the spine, shoulder drop, and muscular pain in the neck and back.

Prosthetic forms are usually positioned in post mastectomy swimsuits or post mastectomy bras. The basic requirements for mastectomy lingerie such as mastectomy bras, mastectomy sleepwear or mastectomy swimsuits is conformance to the natural feel and comfort, as well as size adjustment for different types of uses. It is recognized that the prosthetic forms can differ depending on the type of garment worn by the user.

Conventionally, there exist several types of external breast prosthesis. One type of prosthesis is made of silicone and designed to model natural breast tissue. The prosthesis is designed to weigh the same as the natural breast to help prevent shoulder drop and poor balance. Nonsilicone breast prosthesis is a lightweight breast form made of foam or fiberfill and can be worn right after a mastectomy. It can also be worn during exercise, swimming, and in hot weather.

Still another type is made as an attachable breast, which is fastened to the chest wall using adhesive strips. There also exists a postsurgical soft-form camisole, which is made of a soft, stretchy material with lace elastic straps and is often worn immediately after a mastectomy, lumpectomy (removal of the tumor and some tissue around the tumor), radiation therapy to the chest, or reconstructive breast surgery. A lightweight, removable soft breast form can be placed into a pocket in the camisole. Partial breast prosthesis, also called an equalizer or enhancer, is made of foam, fiberfill, or silicone. It is designed to be worn over a woman's own breast tissue to create a fuller appearance after a part of her breast is removed, such as with a lumpectomy.

It is recognized that the weight of the post mastectomy prosthesis or post mastectomy breast form is critical for comfort. When pockets are sawn in post-mastectomy lingerie, it is important that the prosthesis is kept in proper position during activities. Fashion is of utmost importance when post mastectomy swimsuits, post mastectomy bras, post mastectomy sleepwear and post mastectomy lingerie is designed.

However, conventional prosthetic forms do not allow the user to adjust the shape or size of the prosthesis depending on the type of lingerie or garment being worn by the user. As a result, the patients have to buy several types and shapes of prosthetic forms, which can be expensive. The present invention contemplates elimination of drawbacks associated with conventional prosthetic forms and provision of a prosthetic form that the user can modify by size and weight.

### SUMMARY OF THE INVENTION

It is, therefore, an object of the present invention to provide a breast prosthetic form that a user can modify depending on the desired size and weight.

It is another object of the invention to provide a kit for making a breast prosthetic form.

It is a further object of the invention to provide a kit for making breast prosthetic forms that easily adjusts to the user's garment.

These and other objects of the invention are achieved through a provision of a kit for making a prosthetic breast form to be worn external to a human body that allows a user to make the prosthetic form depending on the desired shape and size of the prosthesis. The kit has at least one, and preferably a plurality of flexible resilient elastic stretchable pouches having an upper opening and a pre-determined quantity of granular filler adapted to simulate weight and volume characteristics of a human breast. A funnel is provided to facilitate depositing of the desired quantity of the granular filler into the pouches.

### BRIEF DESCRIPTION OF THE DRAWINGS

Reference will now be made to the drawings, wherein like parts are designated by like numerals, and wherein

FIG. 1 is a perspective view of the kit for making a breast prosthetic form according to the present invention.

FIG. 2 is a perspective view of the pouches for making the breast prosthetic forms.

FIG. 3 is a schematic view illustrating a cup of a bra having a prosthetic form positioned therein.

FIG. 4 is a cross sectional view of the prosthetic form made with granular material.

FIG. 5 illustrates position of the prosthetic form of the present invention in a bra.

### DETAIL DESCRIPTION OF THE INVENTION

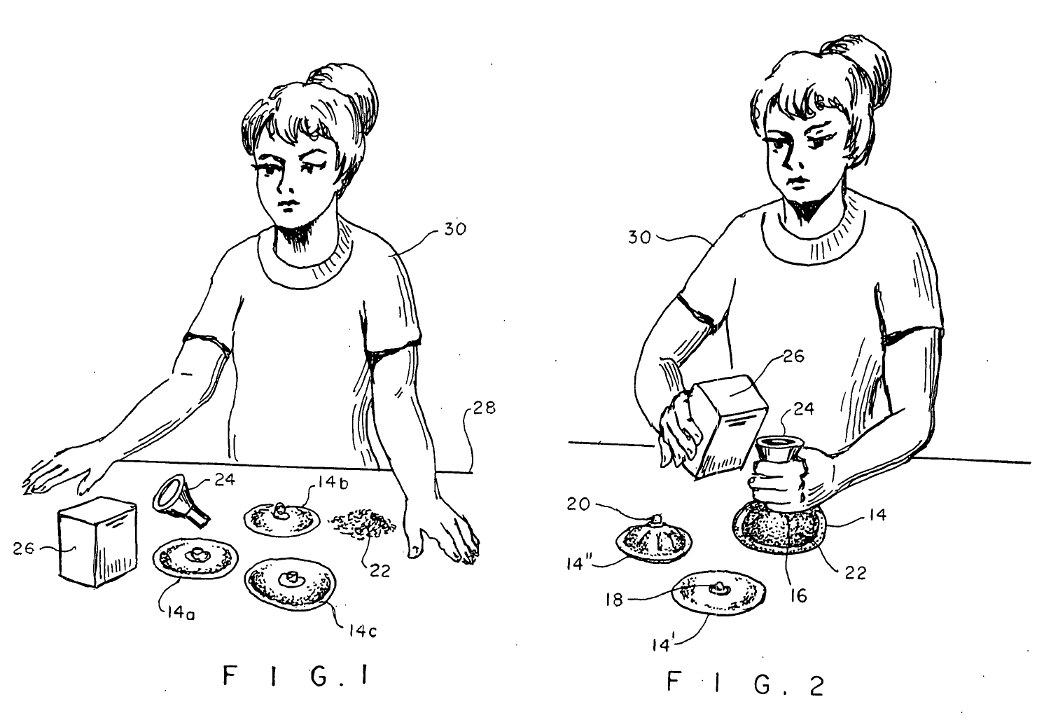

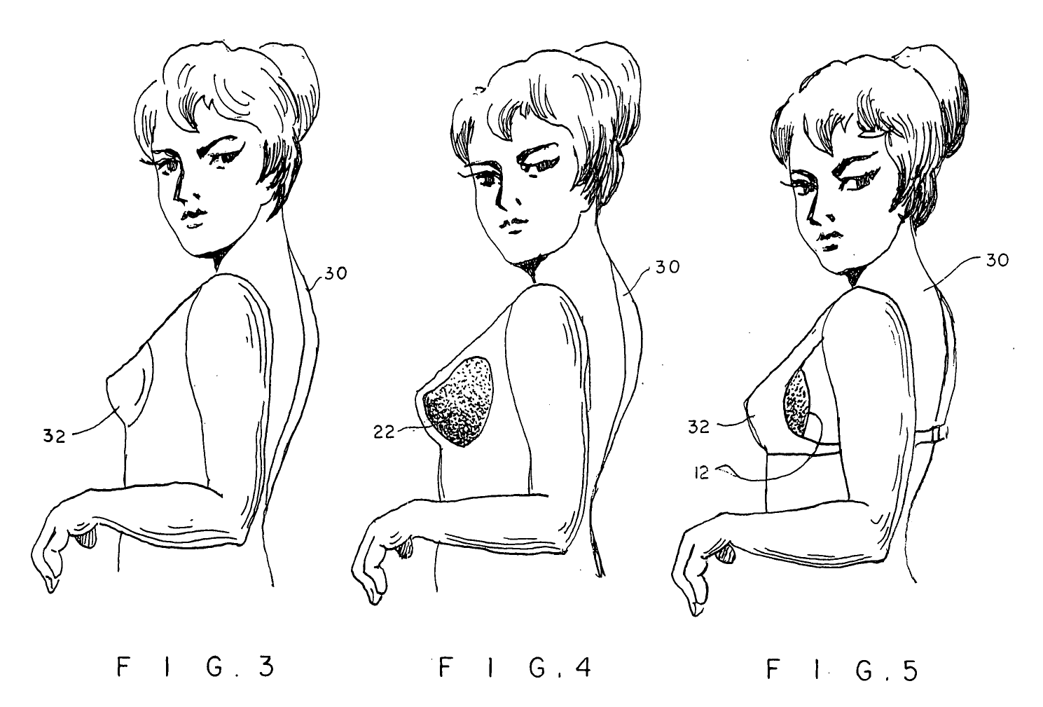

Turning now to the drawings in more detail, FIG. 1 depicts a kit 10 for making a prosthetic form 12 according to the present invention. The prosthetic breast form 12 can be shaped as a full or partial breast form, depending on the desired size and shape. The prosthetic breast form 12 comprises a pouch or pocket-like structure 14 that can be fabricated from a soft, flexible, stretchable elastic material, such as nylon net or other fabrics blended with nylon. Alternatively, the pouch can be made of a composition of nylon and a mixture of spandex, which provides the elasticity and form-fitting that is required for the purposes of this invention.

Variations in the materials used for manufacturing the pouch 14 include microfibers blended with nylon, silk, cotton, or wool. In one aspect of the invention, the pouch 14 is formed from a blend of nylon net material similar to the material of pantyhose. The pouch can be composed of double knit fabric consisting of elastic polyurethane fiber, such as Lycra, and nylon, or cotton and elastic polyurethane fiber, such as a Lycra material. The pouch 14 can be formed in various sizes, and it is envisioned that the kit 10 will contain various sizes pouches, such as for instance pouches 14a, 14b, and 14c shown in FIG. 1. The kit 10 contains at least one and in the preferred embodiments, a plurality of pouches 14. It is envisioned that the pouches 14 can be reused by the customer or user.

Each pouch 14 is formed as a generally continuous tubular or drop-shaped body having a closed bottom 16 and defining an open end or upper opening 18 opposite the closed bottom 16. Of course, other shapes of pouches may be employed as well. For instance, the pouch 14 can be formed using two rectangular panels sewn together to form a closed bottom container or tear-drop shaped to more closely resemble the shape of a human breast.

An empty pouch 14 can collapse, as illustrated at 14′ in the bottom of FIG. 2. When partially lifted by the neck adjacent the upper opening 18, as illustrated at 14″ in FIG. 2, the pouch 14 gradually unfolds and obtains the desired pouch shape. The pouch 14 defines an inner chamber configured to receive a filler material, as will be explained in more detail hereinafter and simulate the shape and size of a natural woman's breast.

A closing means, such as a drawstring, is provided for securing the open end 18. In one aspect of the invention the closing means comprises an elongated strip 20 made of hook-and-loop fastener material. The closing means can be either attached to the pouch a distance from the open end 18 or provided separately as part of the kit 10.

A pre-determined quantity of granular filler 22 material is provided in the kit 10 for depositing into the pouch 14 and forming the prosthetic form 12. The granular filler 22 material can be made from a variety of free-flowing granules, including round and elongated granules. In one of the preferred embodiments, the granules of the granular filler 22 are selected to be between 0.5 mm (0.0197 inch) and 5 mm (0.197 inch), although it is envisioned that in some cases, granules of up to 7 mm (0.276 inch) can be used as well. If elongated granules are used, it is envisioned that the length/width ratio of the granules would not exceed 3. In empirical tests, the filler material was selected to have 1 cup (0.237 liter) of the filler material equal to about 240 cc.

The physical characteristics of the granular filler (granules or beads) 22 are selected to resemble the weight and volume characteristics of a woman's breast. The granular filler material 22 can be made of organic or inorganic matter, natural or artificial, such as for instance polystyrene, micro beads, micro polystyrene, polypropylene pellets, sodium aluminosilicate, millet, rice, flaxseed, and the like. Although FIG. 1 illustrates the filer material as a pile on a support surface 28, it will be understood that the filler material 22 will be placed in a suitable bag (not shown) or separate box 26 to prevent spillage in a container (not shown), in which the kit 10 is packaged.

The kit 10 further comprises a channeling tool, such as a funnel 24 for pouring the fine-grained substance of the filler material 22 into the pouch 14. The funnel 24 can be made from stainless steel, aluminum, plastic, or other suitable non-reactive non-corrosive materials. The kit 10 can come in a box (not shown), wherein the pouches 14, the granular filler 22, and the funnel 24 are packaged.

In operation, the user 30 selects one of the pouches 14 depending on the size of the breast form the user wishes to make. The user then positions the funnel 24 into the upper opening 18 of the selected pouch 14a, 14b, or 14c and deposits the granular filler 22 through the funnel 24 into the pouch 14, as illustrated at 14 in FIG. 2. The user 30 then tightens the drawstring or the elongated strip 20 around the neck of the pouch 14, closing the pouch and preventing the filler granules from escaping from the pouch 14. The user then positions the filled pouch 14, which becomes the prosthetic breast form 12, in a cup 32 of the bra, swimsuit or a pocket of a mastectomy brassiere. Since the pouch is flexible, stretchable and elastic, the prosthetic breast form 12 immediately conforms to the body of the user 30 and to the cup 32 of the bra worn by the user.

If the user changes the garment or the lingerie, the user can use other pouch bodies for making different size forms. Alternatively, the user can open the pouch 14, add more granular material or remove some of the granular filler 22 to accommodate the desired size and mass. These adjustments can be done in the privacy of the user's home without the need for special assistant or technician. If a strong net material is selected for the pouches 14 the pouches can be reused numerous times.

The prosthetic breast form 12 comfortably adjusts to the woman's chest wall and to the mastectomy bra. The prosthetic breast form 12 readily shapes to the contours of the chest wall formed after a surgery. It is envisioned that the granular material and mass thereof can be varied in order to provide the size, shape and weight suitable for matching the woman's natural breast.

Many changes and modifications can be made in the design of the present invention without departing from the spirit thereof. I, therefore, pray that my rights to the present invention be limited only by the scope of the appended claims.

I claim:

1. A kit for making a prosthetic breast form to be worn external to a human body, comprising:
at least one flexible resilient elastic stretchable pouch having an upper opening;
a pre-determined quantity of granular filler adapted to simulate weight and volume characteristics of a human breast;
a means for depositing the granular filler into said at least one pouch; and
a tying means for closing the upper opening after the granular filler is deposited in said at least one pouch.

2. The kit of claim 1, wherein said granular filler comprises a granulated material with granules of between 0.5 mm and 7 mm.

3. The kit of claim 2, wherein the granules are round beads.

4. The kit if claim 2, wherein the granules are elongated beads having length to width ratio of less than 3.

5. The kit of claim 1, wherein the granular filler is made from a material selected from a group consisting of organic, inorganic, natural and artificial materials.

6. The kit of claim 1, wherein the tying means is a drawstring.

7. The kit of claim 1, wherein the tying means is a strip of hook-and-loop fasteners.

8. The kit of claim 1, wherein the means for deposing the granular filler into the pouch comprises a funnel.

9. The kit of claim 1, comprising a plurality of different size pouches.

10. The kit of claim 1, wherein said at least one pouch is formed as a generally continuous tubular body.

11. The kit of claim 1, wherein said at least one pouch is formed as a generally continuous drop-shaped body.

12. The kit of claim 1, wherein said at least one pouch is formed from a material selected from the group consisting of nylon net, spandex, elastic polyurethane fiber, and microfibers blended with nylon, silk, cotton, or wool.

13. A prosthetic breast form configured to be worn external to a human body, the form comprising a flexible resilient elastic stretchable pouch defining an inner chamber and a predetermined quantity of granular filler deposited in the inner chamber, said filler material being adapted to simulate physical characteristics of a natural woman's breast.

14. The prosthetic breast form of claim 13, wherein the pouch comprises a generally continuous tubular body.

15. The prosthetic breast form of claim 13, wherein the pouch comprises a generally continuous drop-shaped body.

16. The prosthetic breast form of claim 13, wherein said pouch is formed from a material selected from the group consisting of nylon net, spandex, elastic polyurethane fiber, and microfibers blended with nylon, silk, cotton, or wool.

17. The prosthetic breast form of claim 13, wherein said granular filler comprises a granulated material with granules of between 0.5 mm and 7 mm.

18. The prosthetic breast form of claim 17, wherein the granules are round beads.

19. The prosthetic breast form of claim 17, wherein the granules are elongated beads having length to width ratio of less than 3.

20. The prosthetic breast form of claim 13, wherein the granular filler is made from a material selected from a group consisting of organic, inorganic, natural and artificial materials.

21. A method of manufacturing a prosthetic breast form to be worn external to a human body, comprising the steps:
providing at least flexible resilient elastic stretchable pouch having an upper opening;
providing a pre-determined quantity of granular filler adapted to simulate weight and volume characteristics of a human breast;
depositing a sufficient quantity of the granular filler into the pouch to achieve a desired size of the prosthetic form to simulate a natural woman's breast; and
closing the upper opening after the granular filler is deposited into the pouch.

22. The method of claim 21, further comprising a step of providing a funnel to facilitate deposit of the granular filler into the pouch.

23. The method of claim 21, wherein said pouch is formed from a material selected from the group consisting of nylon net, spandex, elastic polyurethane fiber, and microfibers blended with nylon, silk, cotton, or wool.

24. The method of claim 21, wherein said granular filler comprises a granulated material with granules of between 0.5 mm and 7 mm.

25. The method of claim 24, wherein the granules are round beads.

26. The method of claim 24, wherein the granules are elongated beads having length to width ratio of less than 3.

27. The method of claim 21, wherein the granular filler is made from a material selected from a group consisting of organic, inorganic, natural and artificial materials.
